(19) United States
(12) Patent Application Publication
(10) Pub. No.: US 2007/0118139 A1
(43) Pub. Date: May 24, 2007

(54) System and method for bone resection

(76) Inventors: Alberto D. Cuellar, Houston, TX (US); Amir Sarvestani, Freiburg (DE); Yashdeep Kumar, Portage, MI (US)

Correspondence Address: MCCRACKEN & FRANK LLP, 200 W. ADAMS STREET, SUITE 2150, CHICAGO, IL, 60606, US

(21) Appl. No.: 11/251,044

(22) Filed: Oct. 14, 2005

(51) Int. Cl. A61F 5/00

(52) U.S. Cl. 606/87

(57) ABSTRACT

A method and systems for performing an osteotomy of a bone using a surgical navigation system without attaching a bone tracking device directly to any portion of the bone that will remain after the osteotomy includes affixing a tracking device to a portion of the bone that will be removed during the osteotomy, determining an anatomical profile of the bone; and performing cuts using positional guidance from the surgical navigation system.

### CROSS REFERENCE TO RELATED APPLICATIONS

Not applicable

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

This invention relates to a method and system for performing bone resections in orthopedic surgeries, such as a total knee arthroplasty, using a surgical navigation system. More particularly, this invention relates to a less invasive technique for attaching tracking devices to boney structure.

2. Description of the Background of the Invention

Many orthopedic surgeries involve making bone cuts or osteotomy. These bone cuts must be made with precision because the implants that are placed over these bone cuts must function in a manner as close to healthy natural anatomy as possible. One current technique for precisely locating the osteotomy utilizes a surgical navigation system that enables the surgeon to guide the surgeon's instruments and tools to the precise location necessary to make the appropriate cuts. Orthopedic surgeons also utilize guides and jigs, either alone or with surgical navigation, to prepare boney structures with the precision necessary to receive an implant that will provide suitable mobility and quality of life to the patient.

One common type of orthopedic surgery is a total knee arthroplasty (TKA). This surgery involves the replacement of the entire knee joint with implants that replicate a healthy knee joint. The preparation of the femur and the tibia for the TKA procedure typically involves multiple bone cuts, each of which should be at an optimum angle for an optimum result. An alternative knee procedure is known as an unicompartmental knee arthroplasty (UKA). This procedure is used where only a portion of either the femur or the tibia is diseased. Only that portion or compartment is removed and a partial implant is inserted to replace the portion removed. One advantage is that the portion of the knee that is not diseased and is still functional will be used along with the implant to provide a more stable knee with potentially less complications. UKA procedures still require careful balancing of the restored portion of the knee so that the restored portion matches the natural portion closely to provide full functionality and mobility.

Surgical navigation systems have been used for surgeries that involve the precise location of instruments relative to a patient's anatomy. These systems were first used in brain operations that require the surgeon accurately place an instrument, probe or similar device in a precise predetermined location in a patient's brain. For orthopedic surgeries, the use of surgical navigation systems has not been as widespread. One reason is that surgical navigation systems require that tracking devices be affixed to the patient in a manner such that the tracking device is unlikely to move relative to the patient during the surgery. In orthopedic surgery, the surgeon will often manipulate the anatomy that is the subject of the surgery to determine range of motion both before and after the procedure. There also are other reasons that the patient and the subject anatomy cannot be fully immobilized during the procedure. This means that the tracking device necessary for surgical navigation must be firmly attached to the patient in a manner so that the tracking device will remain in a fixed relation to the patient's anatomy. The typical method of attachment is to affix these devices directly to a bone that is directly related to the particular surgery. For knee surgery, the location of the femur and the tibia at a minimum need to be tracked. The attachment method is typically a pin or rod type device with a point that is affixed in some fashion directly to the bone. While the risk to the patient is small, any time that the cortex of the bone is disturbed, there is an opportunity for infection or other complication. Also, depending on the size of the pin used, the insertion of these pins can add to patient discomfort and resistance. For some surgeons, these disadvantages outweigh the advantages of precisely locating the boney landmarks to prepare the joint, such as the knee joint, to receive the implants.

### SUMMARY OF THE INVENTION

One aspect of the present invention relates to a method of performing an osteotomy of a bone using a surgical navigation system without attaching a bone tracking device directly to any portion of the bone that will remain after the osteotomy. This method comprises the steps of affixing a tracking device to a portion of the bone that will be removed during the osteotomy, the bone tracking device having a bone reference frame; determining an anatomical profile of the bone; and performing cuts using positional guidance from the surgical navigation system.

A further aspect of the present invention is directed to a method of balancing soft tissue of a joint during a joint arthroplasty using a surgical navigation system after the joint has been prepared to receive implants. This method comprises the steps of first mounting a first trial implant to the prepared joint and a second trial implant to the prepared joint; the first trial implant having a first tracking device attached to the first trial implant, and the second trial implant having a second tracking device attached to the second trial implant. The method also includes displaying joint alignment parameters based on a determined anatomical profile of the joint, and the position of the first tracking device and the second tracking device; and adjusting the soft tissue to balance the joint alignment parameters.

A still further aspect of the present invention relates to system to assist in the performance an osteotomy of a bone for a joint arthroplasty. The system comprises a surgical navigation system having a display; a fixation plate that can be attached to the bone at a location that will be removed during the osteotomy, and the fixation plate having an connection device. The system also includes a bone tracking device directly attached to the fixation plate, the bone tracking device having a bone reference frame, a cutting jig for the bone attached to the fixation plate by the connection device, the cutting jig having a jig tracking device directly attached to the cutting jig, the jig tracking device having a jig reference frame; and a fixation device to fix the cutting jig in place, wherein the cutting jig can be adjusted into position using the display and a previously determined anatomical profile of the bone.

A yet another aspect of the present invention is a method of balancing soft tissue of a knee during a knee arthroplasty using a surgical navigation system after a femur and a tibia have been prepared to receive implants. This method comprises the steps of mounting a trial femoral implant to the prepared femur and a trial tibial implant to the prepared tibia; the trial femoral implant having a femoral tracking device attached to the trial femoral implant, and the trial tibial implant having a tibial tracking device attached to the trial tibial implant; displaying leg alignment parameters based on a previously determined femoral anatomical profile and a previously determined tibial anatomical profile, and the position of the femoral tracking device and the tibial tracking device; and adjusting the soft tissue to balance the leg alignment parameters.

Other aspects and advantages of the present invention will become apparent upon consideration of the following detailed description.

### BRIEF DESCRIPTION OF THE DRAWINGS

FIG. 1 a schematic depiction of a prior art TKA surgery using a surgical navigation system;

FIG. 2 is a side view of one embodiment of a fixation plate and tracking device attached to the distal femur;

FIG. 3 is a top isometric view of the fixation plate of FIG. 2;

FIG. 4 is a side view similar to FIG. 2 showing the use of a pointing device;

FIG. 5 is a side view of one embodiment of a fixation plate and tracking device attached to the proximal tibia;

FIG. 6 is a side view similar to FIG. 2 showing the attachment of one embodiment of a cutting jig to the fixation plate;

FIG. 7 is a side view similar to FIG. 2 showing the attachment of one embodiment of a tracking device to the cutting jig;

FIG. 8 is a screen shot from one embodiment of the surgical navigation system showing the positioning of the cutting jig;

FIG. 9 is a side view similar to FIG. 2 showing one embodiment of affixing the cutting jig in place on the femur;

FIG. 10 is a view similar to FIG. 7 schematically showing transferring the plate reference frame to the jig reference frame;

FIG. 11 is a view similar to FIG. 2 showing the cutting jig attached to a femur that has been partly prepared to receive the implant;

FIG. 12 is a view similar to FIG. 11 showing an optional step of verifying the bone cut;

FIG. 13 is a screen shot showing the location of the bone cut relative to the femur;

FIG. 14 is an isometric view of a further embodiment of a fixation plate;

FIG. 15 is a side view of the device of FIG. 14 in place on a femur;

FIG. 16 is a view similar to FIG. 15 showing the use of a pointer;

FIG. 17 is a view similar to FIG. 15 showing the insertion of a guide pin;

FIG. 18 is a view similar to FIG. 17 with the plate removed;

FIG. 19 is a view similar to FIG. 18 showing the insertion of a screw;

FIG. 20 is a schematic view of a further embodiment of the present invention; and

FIG. 21 is a view similar to FIG. 2 showing a still further embodiment of the present invention.

### DETAILED DESCRIPTION OF THE PREFERRED EMBODIMENTS

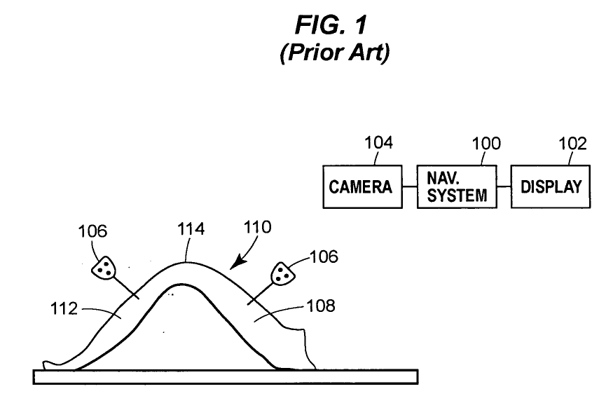

FIG. 1 schematically shows a prior art method of preparing for TKA surgery using a surgical navigation computer 100 that includes a display 102 and a camera 104. The camera 104 is capable of detecting the location and position of tracking devices 106. One tracking device 106 is attached to femur 108 of a patient's leg 110 and the other tracking device 106 is attached to tibia 112 prior to performing TKA surgery on knee 114. As noted above, each tracking device 106 is attached directly to the respective bone, femur 108 or tibia 112, prior to surgery in a location remote from the surgical site for the TKA surgery. The tracking devices 106, or at least the pin necessary to locate the tracking device 106, will remain in place during the surgery and will be removed after the TKA surgery is completed.

The method and system of the present invention will be described in the context of a TKA procedure. However, the method and system of the present invention can also be used to perform any other surgical procedure where sections of the bones of a limb are removed as, e.g., wedge osteotomies in upper and lower extremities, UKA, hip replacement, and other similar procedures.

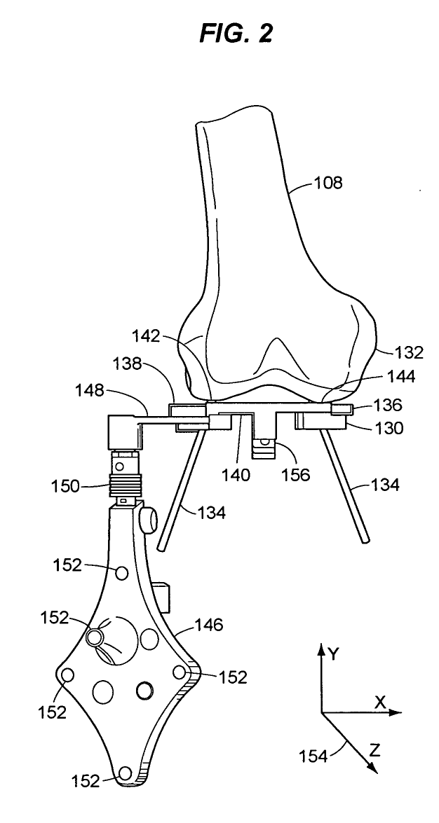

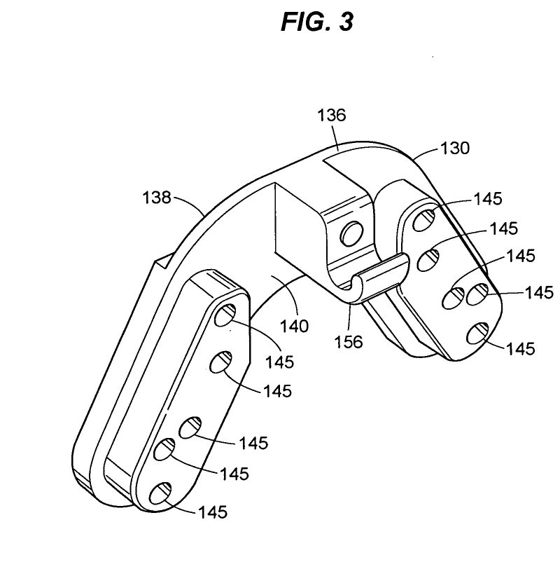

As shown in FIGS. 2 and 3, one embodiment of a fixation plate 130 is attached to a distal portion 132 of the femur 108. The fixation plate 130 is held in place by a plurality of pins 134 that are inserted into the distal portion 132. The fixation plate 130 has a base 136 with a proximal surface 138 and a distal surface 140. Typically, the fixation plate 130 is pinned by pins 134 to medial condoyle 142 and lateral condoyle 144. The pins 134 each pass through one of a series of holes 145 in the base 136. The various holes 145 in the fixation plate 130 to facilitate the correct placement and attachment of the fixation plate 130 to the distal portion 132 of the femur 108. It is not necessary that the fixation plate 130 be positioned in any particular location relative to the subject anatomy, in this case the distal portion 132 of the femur 108. However, it is desirable for certain embodiments of the present invention to locate the fixation plate 130 either on or normal to a particular plane or axis of the subject anatomy. For instance, for the femur 108 the location of the fixation plate 130 parallel to the anterior-posterior axis of the femur will simplify the calculations necessary to properly place a cutting jig or other guide in position relative to the knee 114.

A plate tracking device 146 is removably attached to the base 136 by an arm 148 and a docking device 150. The docking device 150 allows the plate tracking device 146 to be removed when it is no longer needed and also holds the plate tracking device 146 in a fixed position relative to the fixation plate 130. The plate tracking device 146 has a series of LEDs 152 that can be detected by the camera 104 of the surgical navigation computer 100 and has a three dimensional Cartesian reference frame 154. The fixation plate 130 also has an attachment device 156 to enable the fixation plate 130 to be attached to other tools and devices, as will be discussed hereinafter.

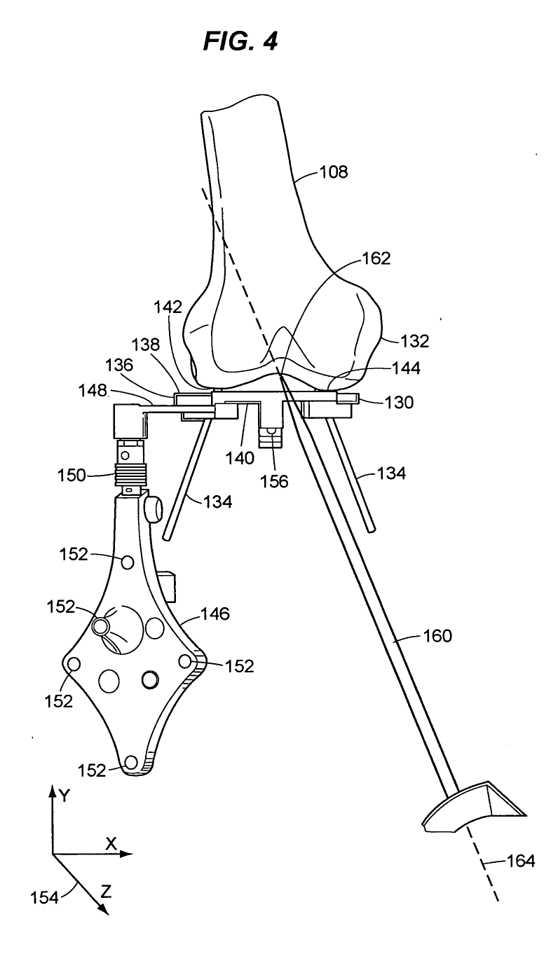

In FIG. 4, a tracked pointer 160 is used to perform a portion of the anatomical survey of the femur 108. The pointer 160 includes a tracking device (not shown) similar to plate tracking device 146. The pointer tracking device can be either integral with the pointer 160 or attached using a similar attachment mechanism as shown in FIG. 2. The pointer 160 also has a pointer tip 162 and a pointer axis 164. Both the location of the pointer tip 162 and the orientation of the pointer axis 164 have been previously calibrated to the pointer tracking device using known technology. The pointer tip 162 is used to trace the surface of the distal portion 132 of the femur 108 and record the shape of the distal portion 132 in memory of the surgical navigation computer 100. Some anatomical structures of interest include the shape of the medial condoyle 142, the shape of the lateral condoyle 144, the anterior-posterior axis of the knee, also known as the Whitesides line, and possibly other features. In addition, by manipulating the femur 108, the location of the plate tracking device 146 can be used by the surgical navigation computer 100 to determine the location of the center of the hip. This is done by taking a large number of readings of the location of the plate tracking device 146 as the femur 108 is manipulated and by calculating the most probable location of the hip center from this cloud of points. Also, the pointer 160 can determine the mechanical axis of the femur 108 using the pointer axis 164. This is accomplished by the surgical navigation system 100 instructing the surgeon first, to locate the femoral center point, this is an anatomical landmark well known to surgeons, with the pointer tip 162, second, to manipulate the pointer 160 so that the pointer axis 164 also points to the hip center, and third, to send a signal to the surgical navigation system 100. At this point, the pointer axis 164 will then be located on the mechanical axis of the femur. In the past, a tracking device that was pinned directly to the femur 108 would have been used to provide the data to determine the location of the center of the hip and the mechanical axis. The structure of the femur 108 to which the fixation plate 130 has been pinned will be removed in preparation for the femoral implant insertion as will be seen later. Using the plate tracking device 146 to perform the anatomical survey will minimize additional intrusions into the femur 108 and will save operating room time because the need for placing extra anchoring devices is eliminated. At this point in the procedure, the surgeon has sufficient information from the anatomical survey done using the pointer 160 and the plate tracking device 146 and from pre-operative scans to determine the location of the modifications that need to be made to the femur 108.

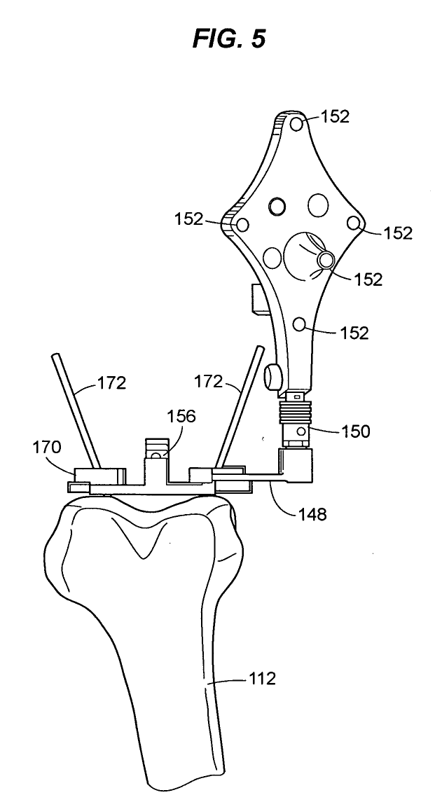

After the femur 108 has been surveyed, the tibia 112 is surveyed. As shown in FIG. 5, a tibial fixation plate 170 is attached to the tibia 112 using pins 172 that are inserted into a portion of the tibia 112 that will be removed in preparation of inserting the tibial implant. In a manner similar to the anatomical survey of the femur 108, an anatomical survey of the tibia 112 is conducted. The locations of the tibia center, the tibial anterior-posterior axis, and both malleoli are determined by manipulating the tibia 112 and/or using the pointer 160.

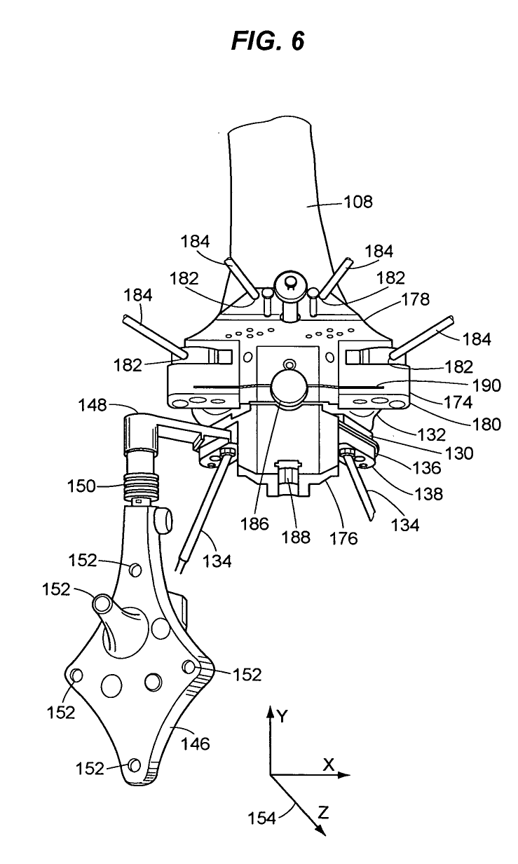

As shown in FIG. 6, a cutting jig 174 is attached to the attachment device 156 of the fixation plate 130 using a connector 176. The connector 176 is capable of adjusting the relative position of the cutting jig 174 to the fixation plate 130. The cutting jig 174 has a body 178, a distal surface 180 and a series of pin apertures 182. A plurality of fixation pins 184 are inserted into some of the pin apertures 182, but the fixation pins 184 are not affixed to the femur 108 at this time.

The connector 176 includes a length adjustment screw 186 to modify the distance between the fixation plate 130 and the cutting jig 174 and an angle adjustment screw 188 to adjust the angle of the distal surface 180 relative to the fixation plate 130. It is also possible to adjust the angle of the distal surface 180 relative to the frontal plane and the sagittal plane of the knee. The cutting jig 174 also has a guide slot 190 that can be used by the surgeon to guide the cuts to be made to the femur 108. For the preparation of the tibia 112, a similar device appropriate for the tibia 112 is attached to the tibial fixation plate 170. The following steps described relative to the femur 108 are also applicable to the tibia 112.

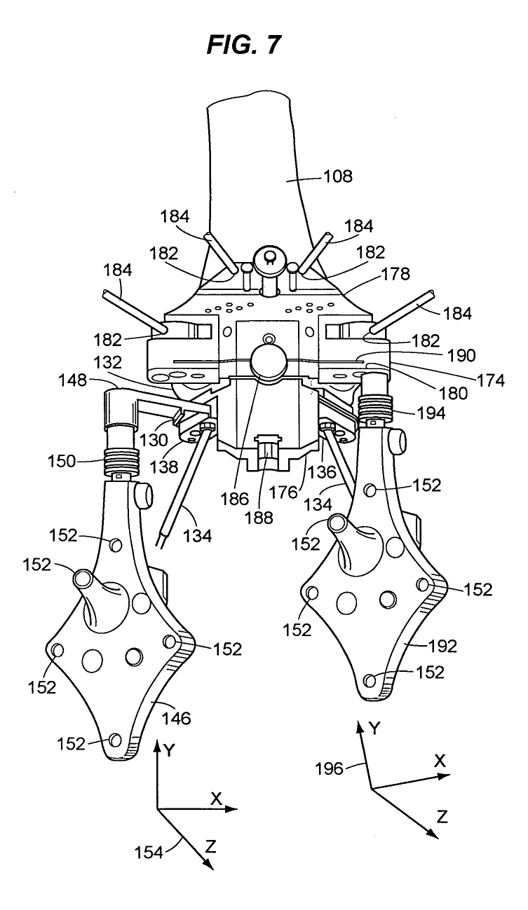

As shown in FIG. 7, a jig tracking device 192 similar to the plate tracking device 146 is attached to the distal surface 180 using an attachment coupling 194. The jig tracking device 192 has a jig reference frame 196. The jig reference frame 196 may be either the same as or different from the plate reference frame 154. At this point, the cutting jig 174 has not been directly affixed to the femur 108 and can be moved to accurately position the cutting jig 174 to the precise location desired by the surgeon.

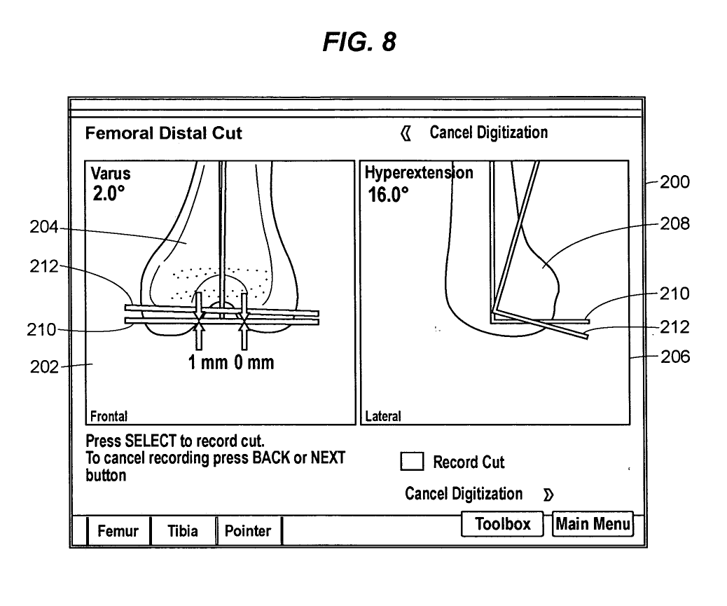

FIG. 8 is a screen shot 200 from the display 102 showing two windows, a first window 202 displaying a digitized frontal view 204 of the femur 108 and a second window 206 displaying a digitized lateral view 208 of the femur 108. Overlaid on the view 204 is a goal axis and plane 210 and a current position of the cutting jig axis and plane 212 based on the location of the jig tracking device 192. In a similar fashion, the second window 206 shows the lateral view of the same goal axis and plane 210 relative to the current position of the cutting jig axis and plane 212, also based on the location of the jig tracking device 192. As shown in the first window 202, the current position of the cutting jig 174 will result in a varus alignment of 2.0°. The second window 206 shows a hyperextension of the knee of 16.0°. By manipulating the adjustment screws 186 and 188 and viewing the display 102, the surgeon can position the cutting jig 174 in the proper position to achieve the desired surgical outcome. There will be similar screens for the tibia 112.

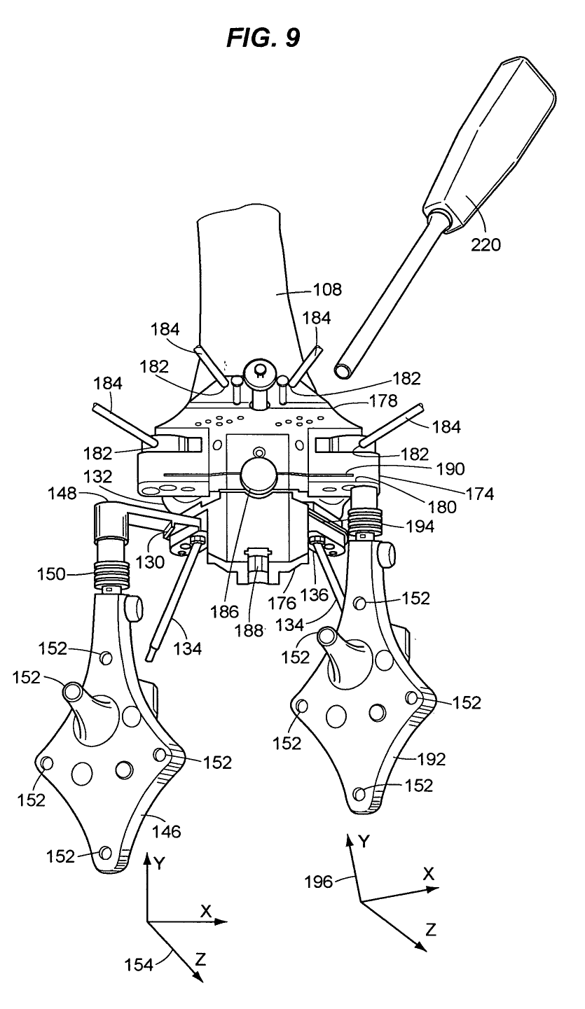

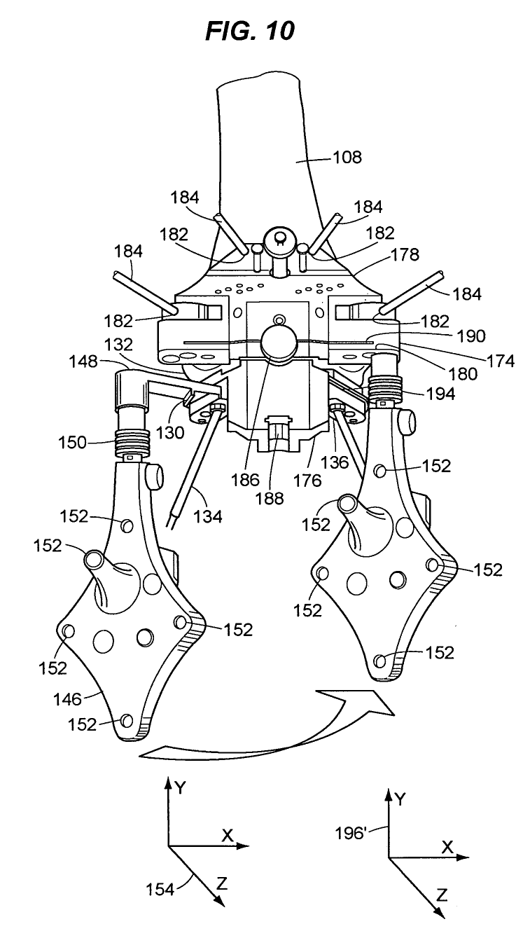

As shown in FIG. 9, when the cutting jig 174 is located in the proper position relative to the femur 108, the fixation pins 184 are then driven into the femur 108 using a tool 220. At this point the cutting jig 174 is then held firmly in position by the fixation pins 184. With the cutting jig 174 now firmly affixed to the femur 108, if the plate reference frame 154 is different from the jig reference frame 196, the surgical navigation computer 100 can replace the jig reference frame 196 with the plate reference frame 154. This is schematically shown in FIG. 10 by arrow 230. After the transfer, the jig reference frame 196 is now a jig reference frame prime 196′. At this point, both the plate reference frame 154 and the jig reference frame prime 196′ are identical and the jig reference frame prime 196′ will provide the same positional data to the surgical navigation computer 100 as the plate reference frame 154 did previously. Also, the surgical navigation computer 100 will adjust all internal references to the jig reference frame 196 to the jig reference frame prime 196′. In this instance, the tracking device 192 is directly attached to the femur 108, but it is attached to the cutting jig 174 without necessitating more invasion to the femur 108 than necessary to firmly affix the cutting jig 174 in position.

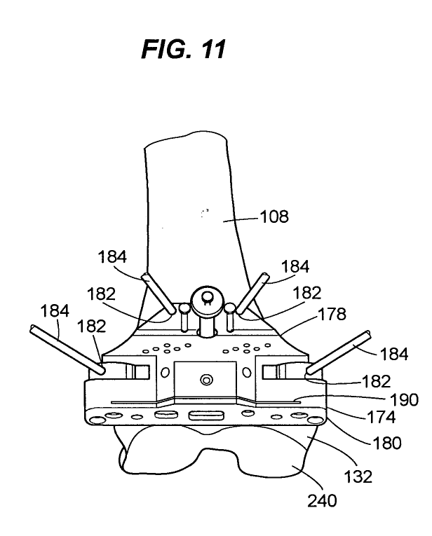

In FIG. 11, the fixation plate 130, the connector 176 and both tracking devices 146 and 192 have been removed leaving only the cutting jig 174 attached to the femur. The view in FIG. 11 is after the modifications have been made to the distal portion 132 of the femur 108 leaving a plateau 240 that is in line with either the guide slot 190 or the distal surface 180 of the cutting jig 174. The bone modifications have been made in a conventional fashion using well known orthopedic tools and procedures

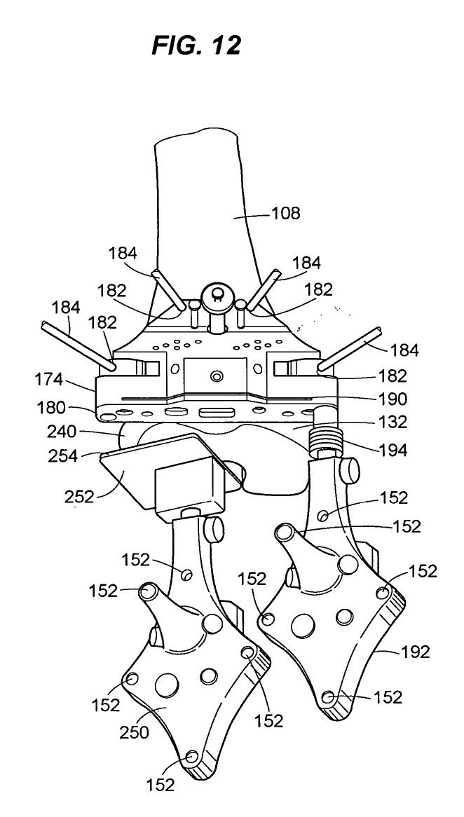

As shown in FIG. 12, the jig tracking device 192 has been reattached to the distal surface 180 of the cutting jig 174. The coupling 194 used to re-attach the jig tracking device 192 ensures that the jig tracking device 192 is reattached in exactly the same position and orientation relative to the cutting jig 174. This re-establishes the jig reference frame prime 196′ for the surgical navigation computer 100. A plane tracking device 250 with a plane probe 252 attached is used to verify the accuracy of the bone modifications made. The plane probe 252 and the plane tracking device 250 have been calibrated so that the surgical navigation computer 100 knows the position and orientation of a plane surface 254 of the plane probe 252 relative to the position and orientation of the plane tracking device 250.

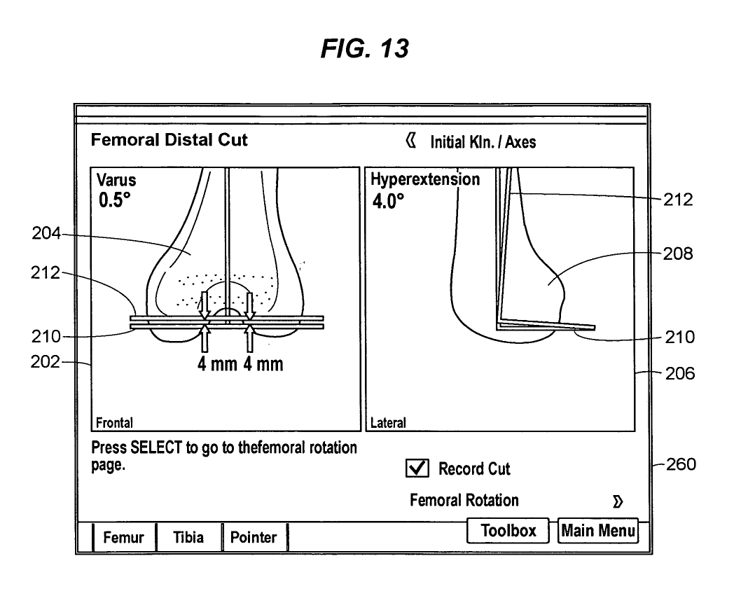

FIG. 13 is a screen shot 260 similar to screen shot 200. The left window 202 shows the relative position of the goal axis and plane 210 and the current axis and plane 208 based on data from the plane probe 252. As shown, the current position as verified by the plane probe 252 will result in a varus alignment of 0.5° and a hyperextension of 4.0°. If this was the surgical goal, then the procedure can proceed to installing the implants in a conventional fashion. If further modifications are needed, they can be made at this point.

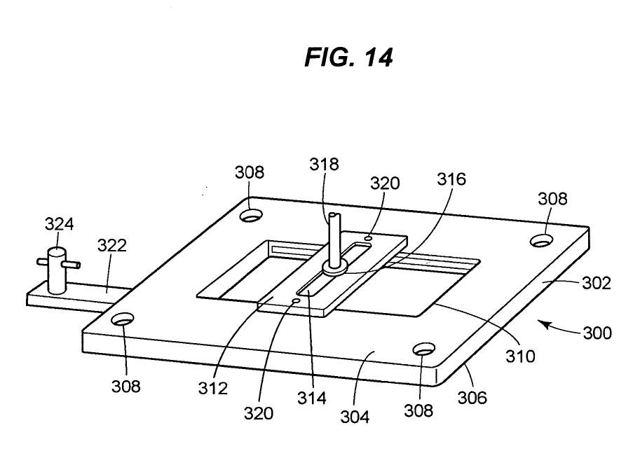

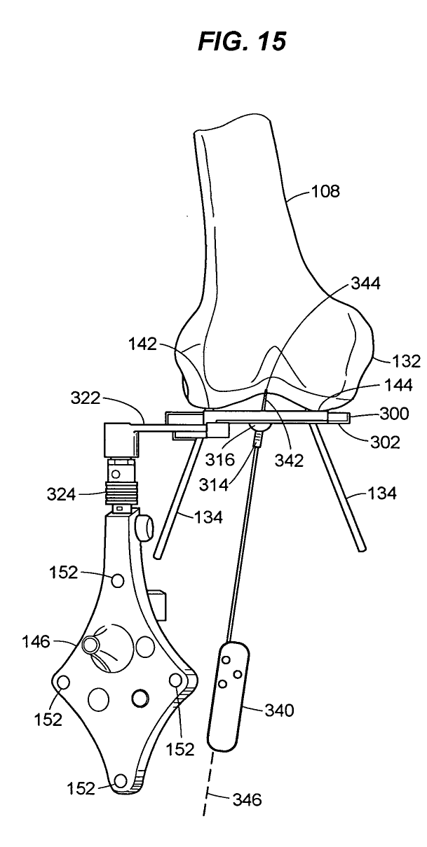

FIGS. 14 and 15 show a further embodiment of a fixation plate 300. The fixation plate 300 has a body 302 with a distal surface 304 and a proximal surface 306. There are a series of apertures 308 (not all are shown) through the body 302 to enable the use of pins 134 to attach the fixation plate 300 to the distal portion 132 of the femur 108 or to the proximal portion of the tibia 112. A slot 310 is in the center of the body 302. The slot 310 has a slide 312 movably mounted therein. With the normal positioning of the fixation plate 300 on the distal portion 132 of the femur 108, the slot 310 will allow the slide 312 to move in the lateral medial direction. The slide 312 also has a slot 314 that is perpendicular to the axis of the slot 310 to move in the anterior-posterior direction. Mounted in the slot 314 is a universal joint 316 that has a cannula 318 passing through the universal joint 316 such that a probe can be inserted into the cannula 318 and pass through the cannula 318 and the slots 310 and 314. The universal joint 316 also is capable of being fixed in position within the slot 314 in order to fix the angle of the cannula 318 relative to the fixation plate 300. In a similar manner, the slide 312 has set screws 320 to fix the location of the slide 312 within the slot 310. In addition, there is an arm 322 with a docking connector pin 324 affixed to the body 302 for mounting of the plate tracking device 146.

The fixation plate 300 will be discussed relative to the use of the fixation plate 300 with the femur 108. The fixation plate 300 can also be used with the tibia 112 in a similar manner. The fixation plate 300 is affixed to the distal portion 132 of the femur 108 using pins 134 affixed through apertures 308. The proximal surface 306 will typically rest on the medial condoyle 142 and the lateral condoyle 144. The precise location and orientation of the fixation plate 300 is not important. However, as discussed above, it may simplify positioning of other equipment relative to the fixation plate 300 to place the fixation plate 300 on a chosen anatomical reference plane or axis such as the anterior-posterior axis. The femur 108 is then surveyed, in part by manipulating the femur 108 so that the center of the hip joint can be located and the mechanical axis of the femur 108 can be determined. This is done in the same manner as described above and in accord with well known principles of anatomy. As described below, an intramedullary screw can be inserted later.

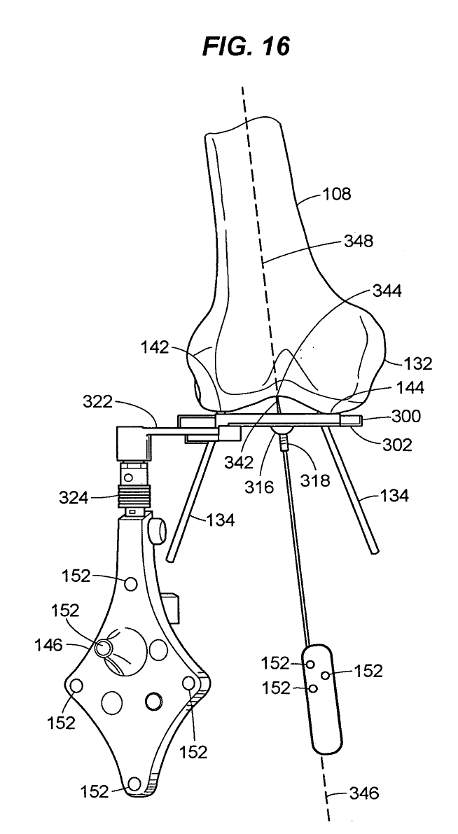

A pointing device 340 is then inserted through the cannula 318 and manipulated so that a pointer tip 342 points to a femoral center point 344. Once the pointer tip 342 is located on the femoral center point 344, the pointing device is manipulated and the slots 310 and 314 and the universal joint 316 allow a pointer axis 346 to move to point to the hip center. When the pointer axis 346 is pointing to the hip center and the pointer tip 342 is at the femoral center point 344 as shown in FIG. 16, the pointer axis 346 is aligned with a mechanical axis 348 of the femur 108. At this point, the set screws 320 and the universal joint 316 are tightened to fix the cannula 318 such that the axis of the cannula 318 is aligned with the mechanical axis of the femur 108.

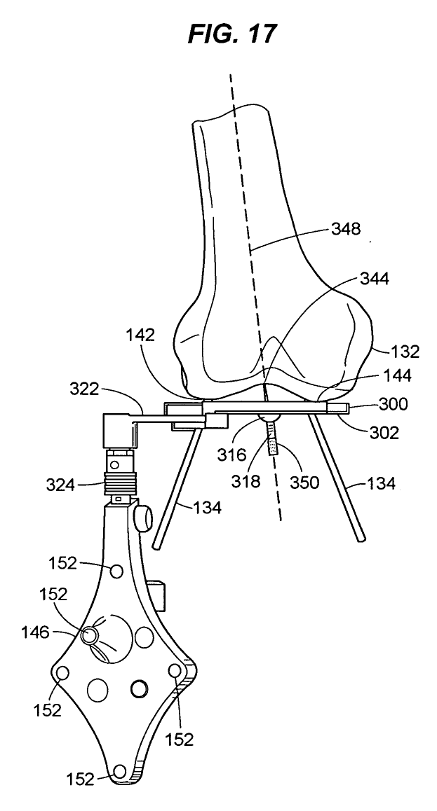

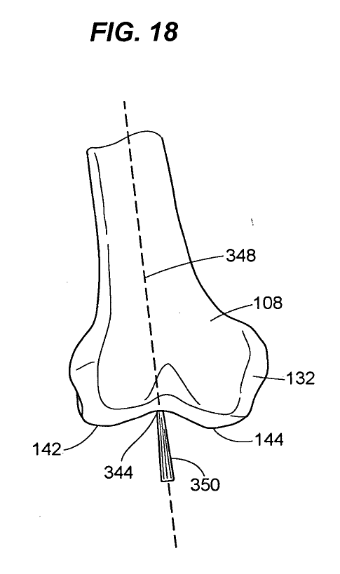

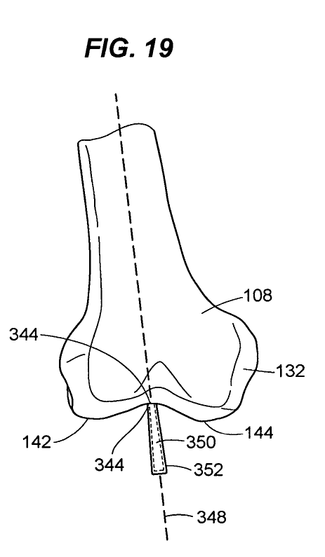

Referring to FIGS. 17 to 19, a guide pin 350 is inserted through the cannula 318 that has been fixed in place and aligned with the mechanical axis 348 of the femur 108 and the guide pin 350 is firmly attached to the femur 108. A hollow self tapping screw 352 is placed over the guide pin 350 and also screwed firmly into position. The guide pin 350 is affixed to a part of the distal portion 132 that will be removed in preparation for the implant. The screw 352 has an exterior surface 354 that is the same diameter as a standard intramedullary rod. At this point, the surgical procedure can proceed by placing the cutting jig 170 over the screw 352 and performing procedures to align the cutting jig 170 without requiring the intrusion into the bone that normally accompanies the insertion of an intramedullary rod.

Because the hollow self tapping screw 352 was set along the mechanical axis of the femur 108 an adjustment of varus/valgus or flexion is not necessary if the cuts are to be made perpendicular to the mechanical axis. In this case, a much simpler jig can be used that only requires the adjustment of the jig's internal/external rotation to properly position the jig.

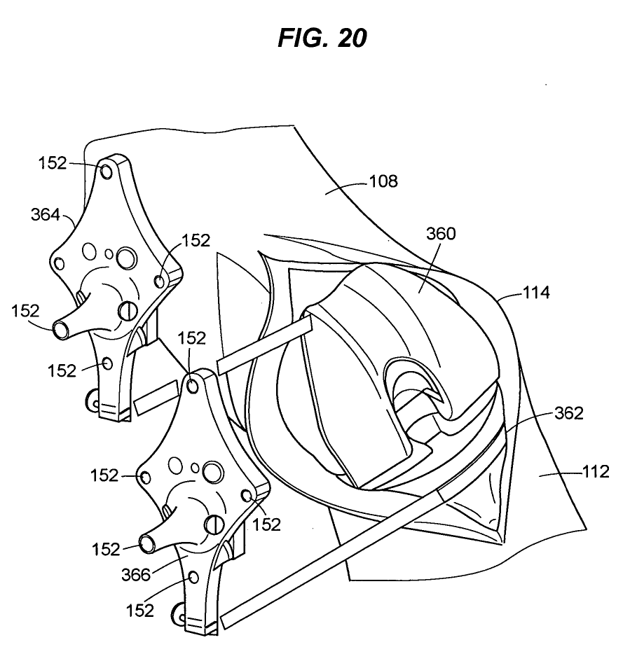

With reference to FIG. 20, after the femur 108 and/or the tibia 112 have been prepared to receive implants, a trial femoral implant 360 is placed onto the prepared femur 108. The trial femoral implant 360 matches the shape and functionality of a final femoral implant that has been chosen based on the preparation of the femur 108 and the sizing requirement of the knee 114. In addition, a trial tibial implant 362 that also replicates the final tibia implant is placed onto the prepared tibia 112. The trial femoral implant 360 has a femoral trial tracking device 364 attached to the trial femoral implant 360 so that the trial femoral tracking device 364 does not interfere with the functionality and flexibility of the knee 114. In a similar manner, a trial tibial tracking device 366 is attached to the trial tibial implant 362. The display 104 will show a screen that includes the anatomical parameters that have been determined for the femur 108 and the tibia 112. The trial femoral tracking device 364 and the trial tibial tracking device 366 will be recognized by the surgical navigation computer 100 and the reference frames for these two tracking devices will be matched to the prior femoral reference plane prime 196′ and the similarly determined tibial reference frame. The knee 114 will be manipulated to test the functionality and stability of the proposed implants using well known techniques. Based on the results of the manipulation, adjustments can be made, if needed, in an appropriate fashion to the soft tissue envelope of the knee 114. Once the functionality of the restored knee 114 has been optimized, the trial femoral implant 360 and the trial tibial implant 362 will be replaced by the final implants that will be affixed to the femur and the tibia using conventional methods.

There is also an alternative method to use the trial femoral implant 360 and the trial tibial implant 362 in a knee 114 that has been prepared without the use of the surgical navigation system 100. In this case, the trial femoral implant 360 and the trial tibial implant 362 are placed in a knee 114 that has been prepared in a conventional fashion. The trial femoral tracking device 364 is attached to the trial femoral implant 360, and the trial tibial tracking device 366 is attached to the trial tibial implant 362. The knee 114 and the femur 108 and the tibia 112 are manipulated to determine the necessary anatomical landmarks and to assist the surgeon in determining if any adjustments are necessary to the soft tissue envelope of the knee 114. Even though the resections made to the femur 108 and the tibia 112 have been made without the assistance of the surgical navigation system 100, the surgical navigation system 110 can assist the surgeon to properly balance the knee 114 using the chosen implants by assisting in making the appropriate releases to the soft tissue envelope surrounding the knee 114.

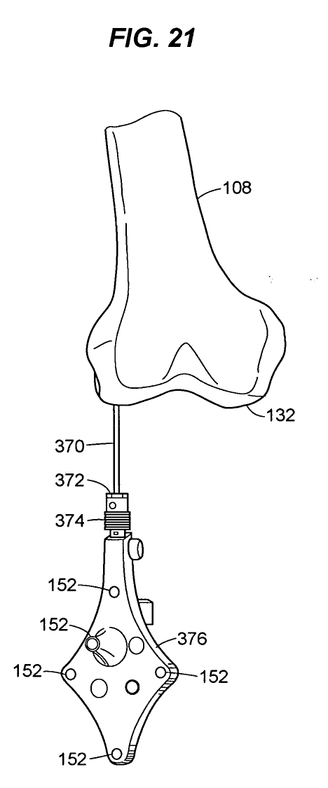

FIG. 21 shows a further embodiment of the present invention. In this embodiment, a pin 370 has a proximal end (not shown) that includes a barb or other conventional connection device to affix the pin 370 to the distal portion 132 of the femur 108. The pin 370 also has a distal end 372. A docking device 374 is affixed to the distal end 372. The docking device 374 is similar to the docking device 150. A tracking device 376 similar to the tracking device 146 can be inserted into the docking device 374. Once the pin 370 with the attached tracking device 376 is affixed to the femur 108, the anatomical profile of the femur can be determined in a manner similar to that described above. The pin 370 is inserted into the femur 108 such that when cuts are made to the femur 108 during a later surgical procedure, the part of the distal portion 132 to which the pin 370 has been attached will have been removed.

### INDUSTRIAL APPLICABILITY

The method and system will assist in the performance of orthopedic surgeries, such as TKA surgery and UKA surgery, with a minimal number of added invasions to the bone.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved. I/We claim:

1. A method of performing an osteotomy of a bone using a surgical navigation system without attaching a bone tracking device directly to any portion of the bone that will remain after the osteotomy, the method comprising the steps of:
affixing a tracking device to a portion of the bone that will be removed during the osteotomy,
the bone tracking device having a bone reference frame;
determining an anatomical profile of the bone; and
performing cuts using positional guidance from the surgical navigation system.

2. Method of claim 1 wherein the tracking device is affixed to the bone using a fixation device.

3. The method of claim 2 that includes the steps of attaching a cutting jig to the fixation device, the cutting jig being adjustable with respect to the fixation plate, the cutting jig having a jig tracking device attached to the cutting jig, the jig tracking device having a jig reference frame;
adjusting the position of the cutting jig using the anatomical profile and the surgical navigation system;
fixing the cutting jig in place; and
cutting the bone using the cutting jig as a guide.

4. The method of claim 3 that includes the step of aligning the fixation device to a relevant anatomical feature of the bone prior to affixing the fixation device in place.

5. The method of claim 4 wherein the fixation device is aligned with respect to the mechanical axis of the bone to provide either rotational or translational adjustment capabilities along the axis.

6. The method of claim 1 wherein the bone is a femur.

7. The method of claim 1 wherein the bone is a tibia.

8. The method of claim 3 that includes the step of transferring the bone reference frame from the bone tracking device to the cutting jig tracking device.

9. The method of claim 1 that includes the step of verifying the osteotomy using a plane probe with a plane probe tracking device.

10. The method of claim 6 wherein the anatomical profile includes the location of the center of the hip joint, the shape of the medial condoyle, the shape of the lateral condoyle and the location of the anterior-posterior axis.

11. The method of claim 10 that includes the step of locating the mechanical axis of the femur by pointing a position tracker having an axis at the center of the femur and then pointing the position tracker at the hip center and identifying the position of the axis of the position tracker.

12. The method of claim 7 wherein the anatomical profile includes the location of the tibia center, the shape of the medial compartment, the shape of the lateral compartment, the location of the anterior-posterior axis, the location of the medial malleolus, and the location of the lateral malleolus.

13. The method of claim 12 that includes the step of locating the mechanical axis of the tibia by pointing a position tracker having an axis at the center of the tibia and then pointing the position tracker at the ankle center and identifying the position of the axis of the position tracker.

14. The method of claim 11 wherein the fixation plate includes a cannula that has a universal joint so that the position tracker can be inserted into the cannula and the universal joint can be locked into position when the position tracker points to the hip center.

15. The method of claim 13 wherein the fixation plate includes a cannula that has a universal joint so that the position tracker can be inserted into the cannula and the universal joint can be locked into position when the position tracker points to the ankle center.

16. The method of claim 14 that includes the step of inserting a guide pin into the femur through the cannula.

17. The method of claim 15 that includes the step of inserting a guide pin into the tibia through the cannula.

18. The method of claim 16 that includes the steps of removing the fixation plate and placing the cutting jig over the guide pin prior to fixing the cutting jig in position.

19. The method of claim 17 that includes the steps of removing the fixation plate and placing the cutting jig over the guide pin prior to fixing the cutting jig in position.

20. The method of claim 16 that includes the steps of removing the fixation plate, placing a self tapping cannualted screw over the guide pin and placing the cutting jig over the screw prior to fixing the cutting jig in position.

21. The method of claim 17 that includes the steps of removing the fixation plate, placing a self tapping cannualted screw over the guide pin and placing the cutting jig over the screw prior to fixing the cutting jig in position.

22. The method of claim 1 that includes the steps of inserting a trial implant that has an implant tracking device attached to the implant into the prepared bone.

23. The method of claim 1 wherein the tracking device is directly attached to the portion of the bone that will be removed.

24. A method of balancing soft tissue of a joint during a joint arthroplasty using a surgical navigation system after the joint has been prepared to receive implants, the method comprising the steps of:
mounting a first trial implant to the prepared joint and a second trial implant to the prepared joint; the first trial implant having a first tracking device attached to the first trial implant, and
the second trial implant having a second tracking device attached to the second trial implant;
displaying joint alignment parameters based on a determined anatomical profile of the joint,
and the position of the first tracking device and the second tracking device; and
adjusting the soft tissue to balance the joint alignment parameters.

25. The method of claim 24 wherein the determined joint anatomical profile is determined after the first trial implant and the second trial implant are placed in the joint.

26. The method of claim 24 wherein the determined joint anatomical profile is determined before the first trial implant and the second trial implant are placed in the joint.

27. The method of claim 24 wherein the joint is the knee.

28. The method of claim 27 wherein the anatomical profile includes the determination of the location of the hip center and the malleoli.

29. The method of claim 27 wherein the anatomical profile includes the determination of the location of the hip center, the knee center, the anterior-posterior axis of the femur, the tibia center, the anterior-posterior axis of the tibia, and the malleoli.

30. The method of claim 27 wherein the leg alignment parameters include varus/valgus, flexion/extension, rotation, medial gap, and lateral gap

31. The method of claim 26 wherein there is a known relation between a first reference frame for the first tracking device and a prior first reference frame used to prepare the joint and between a second reference frame for the second tracking device and a prior second reference frame used to prepare the joint.

32. The method of claim 31 wherein the known relation is identity.

33. The method of claim 26 wherein the previously determined joint anatomical profile was determined before the joint was prepared to receive the implants.

34. A system to assist in the performance an osteotomy of a bone for a joint arthroplasty comprising:
a surgical navigation system having a display;
a fixation plate that can be attached to the bone at a location that will be removed during the osteotomy, the fixation plate having an connection device;
a bone tracking device directly attached to the fixation plate, the bone tracking device having a bone reference frame;
a cutting jig for the bone attached to the fixation plate by the connection device, the cutting jig having a jig tracking device directly attached to the cutting jig, the jig tracking device having a jig reference frame; and
a fixation device to fix the cutting jig in place;
wherein the cutting jig can be adjusted into position using the display and a previously determined anatomical profile of the bone.

35. The system of claim 34 wherein the bone reference frame replaces the jig reference frame in the surgical navigation system after the cutting jig has been fixed into place.

36. The system of claim 34 that includes a plane probe with a plane probe tracking device to verify modifications to the joint using the cutting jig.

37. The system of claim 34 wherein the joint is a knee and the bone is a femur and the previously determined anatomical profile includes the location of the center of the hip joint, the shape of the medial condoyle, the shape of the lateral condoyle and the location of the anterior-posterior axis of the femur.

38. The system of claim 34 wherein the fixation plate includes a cannula that has a universal joint so that a position tracker can be inserted into the cannula and the universal joint can be locked into position when the position tracker points to a mechanical axis of the bone.

39. The system of claim 38 that includes a guide pin that can be inserted into the femur through the cannula.

40. The system of claim 39 includes a self tapping cannulated screw that can be placed over the guide pin and that has an exterior diameter so that the cutting jig can be placed over the screw prior to fixing the cutting jig in position.

41. The system of claim 34 wherein the joint is a knee and the bone is a tibia and the previously determined anatomical profile includes the location of the center of the ankle, the shape of the medial compartment, the shape of the lateral compartment, and the location of the anterior-posterior axis of the tibia.

42. A method of balancing soft tissue of a knee during a knee arthroplasty using a surgical navigation system after a femur and a tibia have been prepared to receive implants, the method comprising the steps of:
mounting a trial femoral implant to the prepared femur and a trial tibial implant to the prepared tibia; the trial femoral implant having a femoral tracking device attached to the trial femoral implant, and the trial tibial implant having a tibial tracking device attached to the trial tibial implant;
displaying leg alignment parameters based on a previously determined femoral anatomical profile and a previously determined tibial anatomical profile, and the position of the femoral tracking device and the tibial tracking device; and
adjusting the soft tissue to balance the leg alignment parameters.

43. The method of claim 42 wherein the femoral tracking device has a known relation to the femoral anatomical profile and the tibial tracking device has a known relation to the tibial anatomical profile.

44. The method of claim 42 wherein the anatomical profile includes the determination of the location of the hip center and the malleoli.

45. The method of claim 42 wherein the anatomical profile includes the determination of the location of the hip center, the knee center, the anterior-posterior axis of the femur, the tibia center, the anterior-posterior axis of the tibia, and the malleoli.

46. The method of claim 42 wherein the leg alignment parameters include varus/valgus, flexion/extension, rotation, medial gap, and lateral gap

47. The method of claim 42 wherein there is a known relation between a femoral reference frame for the femoral tracking device and a prior femoral reference frame used to prepare the femur and between a tibial reference frame for the tibial tracking device and a prior tibial reference frame used to prepare the tibia.

48. The method of claim 47 wherein the known relation is identity.

49. The method of claim 42 wherein the previously determined femoral anatomical profile and a previously determined tibial anatomical profile were determined before the femur and tibia were prepared to receive the implants.
